(19) United States
(12) Patent Application Publication
(10) Pub. No.: US 2011/0207175 A1
(43) Pub. Date: Aug. 25, 2011

(54) MULTI-CULTURE BIOREACTOR SYSTEM

(75) Inventors: Marwan El-Sabban, Odense M (DK); Steen Sindet-Pedersen, London (GB)

(73) Assignee: MC2 Cell ApS, Hellerup (DK)

(21) Appl. No.: 13/122,933

(22) PCT Filed: Oct. 2, 2009
(86) PCT No.: PCT/EP09/62867

§ 371 Date: May 10, 2011

Related U.S. Application Data
(60) Provisional application No. 61/102,992, filed on Oct. 6, 2008.

(51) Int. Cl. C12P 21/00

(52) U.S. Cl. 435/70.3

(57) ABSTRACT

There is provided co-culture bioreactor systems that can maintain stem cells and differentiated cell types in physically isolated environments but can allow biochemical communication between these cells. For instance, a co-culture bioreactor system of the present invention can include a first culture chamber that defines a first inlet and a first outlet such that fluid can flow through the culture chamber. The system can also include a second culture chamber defining a second inlet and a second outlet allowing a second fluid flow through this second chamber. The system can also include a semi-permeable membrane. The semi-permeable membrane can be located between the first culture chamber and the second culture chamber.

### BACKGROUND OF THE INVENTION

The ability to culture in vitro viable three-dimensional cellular constructs that mimic natural tissue has proven very challenging. One of the most difficult of the many problems faced by researchers is that there are multiple dynamic biochemical interactions that take place between and among cells in vivo, many of which have yet to be fully understood, and yet the complicated in vivo system must be accurately modelled if successful development of engineered tissues in vitro is to be accomplished.

Many existing co-culture systems are simple well plate designs that are static in nature and do not allow for manipulation of the local environment beyond the gross chemical inputs to the system. As such, the development of more dynamic co-culture systems has become of interest. However, known dynamic systems, similar to the static systems, often provide only a single source of nutrients/growth stimulants/etc. to all of the cell types held in the system.

Moreover, the different cell types that are co-cultured in both static and dynamic systems are usually maintained in actual physical contact with one another, preventing the development of an isolated cell population, and also limiting means for better understanding the biochemical communications between the cell types during growth and development.

While several tissue engineering breakthroughs have been made, there remain two important challenges to further progress in generating laboratory-grown tissues and organs: (1) the refinement of polymer scaffolding that mimics the organ architecture, and also supports the growth of appropriate stem cells; and (2) an abundant source of stem cells, i.e. those cells having the potential to proliferate and become fully specialized. Such cells, for example, can form bone, cartilage, muscle or fat, depending on the exact nature of their environment. Currently, most, if not all, organs and tissues made in the laboratory are generated using stem cells of animal or in some cases undefined human origin. Unfortunately, tissues made in this manner have very limited clinical use, primarily because they, like donor tissues and organs, are frequently rejected by the recipient's immune system. A scientifically sound and cost effective strategy to circumvent this problem is to use stem cells isolated from the intended tissue recipient.

Recent advances in cellular and molecular biology have created a window of opportunity for the successful isolation of stem cells from embryonic tissue, adult bone marrow, peripheral and umbilical cord blood.

Tissues and organs consist of specialized living cells arranged within a complex structural and functional framework of extracellular matrix (ECM). The great diversity observed in ECM composition contributes enormously to the properties and function of each organ and tissue: the rigidity and tensile strength of bone, the resilience of cartilage, the flexibility and hydrostatic strength of blood vessels, and the elasticity of skin, are examples of how different ECM compositions contribute to tissue function. Equally important is role of ECM during growth, development, and wound repair, where it provides a reservoir for soluble signalling molecules, and through its own dynamic composition, a source of additional signals to migrating, proliferating, and differentiating cells. These molecules are often referred to as soluble factors.

Artificial substitutes for ECM, called scaffolds, can consist of natural or synthetic polymers, or both, and have been used successfully alone and in combination with cells and soluble factors to induce tissue formation or promote tissue repair. Cells are also central to many tissue engineering strategies, and significant efforts have been made to identify and propagate pluripotent stem cells, to identify signaling events important for proper differentiation, and to identify ideal micro-environments for maximum cellular function. These efforts that have led to a convergence of research in bioengineering, biomaterials, ECM, cell growth and differentiation, and soluble factors that control cell fate.

The coordinated function of many cell types is regulated by the integration of extracellular signals derived from soluble factors such as growth factors, and insoluble molecules of the extracellular matrix (ECM). Indeed, accumulating data suggests that cellular behavior (for example growth, differentiation and cell migration) is regulated by the converging down-stream signaling pathways of receptors for growth factors and ECM molecules. These findings have reinforced the importance of scaffold's composition and structure in controlling cellular responses in vitro and in vivo and provided a solid scientific foundation for the development of the new generation of biomaterials.

Based thereon such stem cells may therefore ultimately be used as a renewable source of cells that differentiate into a variety of tissue cells useful for treating a number of diseases and deficiencies. One important use is the treatment of neurological diseases such as Parkinson's disease (“PD”). Unfortunately, neural stem cells are not a particularly abundant source because they reside deep in the brain, severely constraining accessibility for harvesting. Conversely, bone marrow (BM) stem cells are more abundant and accessible. The ease with which bone marrow stem cells are harvested by simple marrow aspiration, makes them excellent candidates for therapeutic use.

BM comprises a number of stem cell types. Best known among these are hematopoietic stem cells (HSCs) and marrow stromal cells (MSCs). In normal mammals, HSCs give rise to blood cells whereas MSCs give rise to cell types that populate other tissues and sites such as cartilage or bone, hematopoietic supportive stromal cells and fat. Recent studies have suggested that these BM stem cells can, under appropriate conditions, differentiate into additional cell types such as cardiac myocytes, liver cells, and skeletal muscle cells.

Additionally, BM stem cells have been shown to have the potential for generating neurons (Sanchez-Ramos et al. Exp. Neurol. 164 247-256 (2000), Woodbury et al. J: Neurosci. Res. 62: 364-370 (2000), Mezey et al. Science 290: 1779-1782 (2000), Brazelton et al. Science 290: 775-1779 (2000). Chopp's group has investigated the use of human MSCs (hMSCs) to treat rats subjected to strokes. Li Y et al., Neurology, 2002, 59: 514-523, tested the effect of intravenously administered hMSCs on neurologic functional deficits after stroke. Treatment with hMSC resulted in significant recovery of function at 14 days compared with control rats with ischemia. Neurologic benefit resulting from this hMSC treatment appeared to derive from the increase of growth factors in the ischemic tissue, the reduction of apoptosis in the penumbral zone of the lesion, and the proliferation of endogenous cells in the subventricular zone. In a later publication from the same group, Chen X et al., J Neurosci Res, 2002, 69: 687-691, investigated the temporal profile of various growth factors including brain-derived neurotrophic factor (BDNF), nerve growth factor (NGF), vascular endothelial growth factor (VEGF), basic fibroblast growth factor (bFGF), and hepatocyte growth factor (HGF), within cultures of human MSCs (hMSCs) conditioned with cerebral tissue extracts from traumatic brain injury (TBI). hMSCs in such cultures responded by producing more BDNF, NGF, VEGF, and HGF, supporting the notion that transplanted hMSCs provide therapeutic benefit in part via a responsive secretion of an array of growth factors that can foster neuro-protection and angiogenesis.

Laboratory grown cells derived from a several stem cell types, including BM-derived stem cells, may be a desirable source of transplantable material for grafting into brains of individuals suffering from neurological disorders.

To induce stem cells to differentiate, it is desirable to identify the right combination of molecules, their relative abundance and cell-culture conditions to (a) support survival and/or self-renewal of undifferentiated cells in culture and (b) stimulate them to become committed to a desired cell lineage. Such cells may then be implanted into an appropriate site in vivo to complete their growth and differentiation program.

The process of HSC (or other stem cell) differentiation into particular progeny in vitro requires the action of many factors, including growth factors, extracellular matrix

(“ECM”) molecules and components, environmental stressors and direct cell-to-cell interactions. The appropriate agents that will enhance or direct stem cell differentiation along a particular path, however, may be difficult to predict.

For example, when human “leukemia inhibitory factor” (hLIF) was added to cultures of human MSCs, these cells developed fibroblastic morphologies (Sanchez-Romos et al.). The same protein, however, had been shown to be essential for maintaining mouse ES cells in an undifferentiated state (Sanchez et al., 2000). This illustrates the difficulty in knowing in advance the effect of a particular molecule on a particular cell type.

As appears from the above it is desirable to provide an extracellular matrix and soluble factors, perfectly reflecting the composition of the extracellular matrix of the tissue into which the stem cells are supposed to differentiate. Meanwhile the prior art does not teach a method by which a human-based extracellular matrix and paracrine factors may be produced without excising tissue from humans.

What is needed in the art is a method for co-culturing stem cells and cells derived from a desired tissue in a dynamic environment in which the stem cells and desired cell types can communicate biochemically, and yet can be separated physically.

### SUMMARY OF THE INVENTION

In one aspect, the present invention is directed to co-culture bioreactor systems that can maintain stem cells and differentiated cell types in physically isolated environments but can allow biochemical communication between these cells. For instance, a co-culture bioreactor system of the invention can include a first culture chamber that defines a first inlet and a first outlet such that fluid can flow through the culture chamber. The system can also include a second culture chamber defining a second inlet and a second outlet allowing a second fluid flow through this second chamber. The system can also include a semi-permeable membrane. The semi-permeable membrane can be located between the first culture chamber and the second culture chamber. The semi-permeable membrane can have a porosity so as to allow passage of cellular expression products through the membrane, but so as to prevent passage of the cells held in either chamber through the membrane. In a preferred embodiment, the semi-permeable membrane is formed of a material, for example polypropylene, which encourage cellular attachment to the membrane.

The systems can also be capable of incorporating additional culture chambers that can be in biochemical communication with one or both of the other two culture chambers. For instance, a third chamber can house cells that can be in biochemical communication with the first culture chamber, optionally with a semi-permeable membrane separating the first and third chambers, though this aspect is not a requirement of the system.

The bioreactors can be used for growth and development of isolated cells in various different applications. For instance, three- dimensional cellular constructs can be formed including only the cells that are isolated in one of the culture chambers of the reactor system. In one embodiment, a culture chamber can be seeded with undifferentiated cells, and the method can include triggering differentiation of the cells via the biochemical triggers provided from the cells of the second culture chamber.

In a preferred embodiment the present invention is directed at the production of extracellular matrix components and soluble factors based on cultivating differentiating stem cells in order to induce their production of extracellular matrix. The present inventors have surprisingly found that human extracellular matrix extracts produced by differentiating stem cells largely reflect the corresponding in vivo composition of the extracellular matrix, and can thus later function as optimized differentiation environment for progenitors and stem cells, which will then differentiate into the cell types that are normally harbouring the tissue having this extra cellular matrix composition.

According to the invention there is provided a method for producing a human-based extracellular matrix extract and soluble factors of a desired human tissue, comprising,
seeding human stem cells in a culture vessel in a first culture chamber,
providing cells of desired tissue to a second culture chamber, said cells of desired tissue comprising extracellular matrix resembling the extracellular matrix of the desired tissue,
maintaining said stem cells and said cells of desired tissue in a physically isolated state from one another; and allowing biochemical communication between said first cell type and said second cell type,
culturing the stem cells in order to differentiate them into cells of the desired tissue, whereby the cells are stimulated to synthesise, secrete and organize extracellular matrix;
continued culturing of the cells until the cells have been differentiated into cells of the desired tissue and have synthesized extracellular matrix and soluble factors, and
removing the cells to obtain the extracellular matrix extract and soluble factors of the desired tissue.

Accordingly, the present invention provides a method for manufacturing extracellular extracts and soluble factors ex vivo, wherein the composition of the extracts resembles the human in vivo composition. Such extracts are very suitable for differentiating stem cells into the cells of a tissue of interest.

As the human stem cells differentiate they will produce an extracellular matrix layer or body, which predominantly contains differentiated human cells along with the extracellular matrix produced by them-selves. Since the in vivo composition of the extracellular matrix is achieved when the stem cells are fully differentiated into the cells of the tissue of interest it is necessary to cultivate the stem cells for generally at least 14 days. In any event the cells are assessed before harvesting the extracellular matrix; as a general rule at least 90% of the cells should be fully differentiated before preparing the extracellular matrix.

The present invention is further directed to the use of extracts according to the present invention for differentiation of stem cells. For example the extracts may be used for differentiation of mesenchymal stem cells into at least one type of tissue, in particular into bone or cartilage tissue. Likewise the extracts may be used for differentiation of hematopoietic stem cells into hematopoietic progenitor cells.

In one aspect, the present method is used to manufacture extracellular matrix pertaining to liver tissue by seeding and culturing hepatocytes for stimulated synthesis of extracellular matrix extract resembling the composition of the matrix in liver tissue. This ECM extract can be directly applied to adult stem cells to differentiate them into hepatocytes.

### BRIEF DESCRIPTION OF THE DRAWINGS

A full and enabling disclosure of the present invention, including the best mode thereof, to one of ordinary skill in the art, is set forth more particularly in the remainder of the specification, including reference to the accompanying figures, in which:

FIG. 1 is a schematic diagram of an embodiment following assembly such that the two cell modules are adjacent and allow biochemical communication between cells held in the two adjacent modules. In accordance with the figure there is provided regular growth media (1), BMMSC cultured on Human liver cells ECM (2), human liver cells conditioned media (3), and human liver cells cultured under the insert (4).

FIG. 2 shows the effect of ECM dilution on the expression of Hepatocyte Differentiation Markers. In accordance with the figure there is provided BMMSCs (lane 1), BMMSC on xμl human liver cells ECM (lane 2), BMMSC on x/4 μl human liver cells ECM (lane 3).

FIG. 3 demonstrates the induction of P450 expression in ECM differentiated BMMSC. In accordance with the figure there is provided BMMSC untreated (lane 1), BMMSC on human liver cells ECM (lane 2), BMMSC on human liver cells ECM treated for 1 hr (lane 3), and BMMSC on human liver cells ECM treated for 4 hr (lane 4).

FIG. 4 shows the morphology of differentiated BMMSC cultivated on human liver cell ECM.

### DETAILED DESCRIPTION OF THE INVENTION

Reference will now be made in detail to various embodiments of the invention, one or more examples of which are set forth below. Each embodiment is provided by way of explanation of the invention, not limitation of the invention. In fact, it will be apparent to those skilled in the art that various modifications and variations may be made in the present invention without departing from the scope or spirit of the invention. For instance, features illustrated or described as part of one embodiment, may be used in another embodiment to yield a still further embodiment.

In one aspect, the present invention is directed to multi-chambered co-culture systems. The systems of the invention can be utilized for the growth and development of isolated cells of one or more cell types in a dynamic in vitro environment more closely resembling that found in vivo. For instance, the multi- chambered systems of the present invention can allow biochemical communication between cells of different types while maintaining the different cell types in a physically separated state, and moreover, can do so while allowing the cell types held in any one chamber to grow and develop with a three-dimensional aspect. In addition, the presently disclosed devices and systems can allow for variation and independent control of environmental factors within the individual chambers. For instance, the chemical make-up of a nutrient medium that can flow through a chamber as well as the mechanical force environment within the chamber including the perfusion flow, hydrostatic pressure, and the like, can be independently controlled and maintained for each separate culture chamber of the disclosed systems.

In one application undifferentiated stem cells can be located in a first chamber, and one or more types of feeder cells can be located in adjacent chamber(s).

Current engineered living tissue constructs are not completely cell assembled and must rely on either the addition or incorporation of exogenous matrix components or synthetic members for structure or support, or both.

The culture media or extracts of the present invention exhibit many of the native features of the tissue from which their cells are derived.

### Definitions

“Resembles” as used herein means there is physical, compositional, structural, functional, phenotypic or other similarities between the materials or systems being compared, such that the objects are substantially equivalent. “Substantially equivalent” means that visible, microscopic, physical, functional, and other observations and assays do not easily or significantly distinguish the materials or systems. An easy or significant distinction would, for example, be a functional difference, a physical difference, a compositional difference, a structural difference immediately apparent, or easily detectable with standard assays and observational techniques such as staining, microscopy, antibodies, etc. “Extracellular Matrix” (ECM) or “Cell Derived Matrix” (CDM) or Cell-produced Matrix as used interchangeably herein means a cell-derived secreted substance produced by and/or secreted from cells into the extracellular space. The ECM/CDM provides a growth template for any cell type to grow, differentiate, and produce tissue.

The ECM allows cell attachment and cell migration, and promotes cell differentiation. The ECM also aids the formation of new tissue of a desired or existing cell type. As used herein “Cell-Produced Matrix, also called Cell-Derived Matrix (CDM)” also means a 3-dimensional ECM (or matrix) structure that has been completely produced and arranged by cells (or entities) in vitro.

“Construct” as used herein means a physical structure with mechanical properties such as a matrix of scaffold. Construct encompasses both autogenic living scaffolds and living tissue matrices, ex-vivo cell-produced tissue and cell-derived matrix. “Cell-derived” as used herein means that the source for the material, body, or component is a cell or a collection of cells.

“Ex-vivo Cell-produced Tissue (ECT)” as used herein means, a functional tissue comprising one or more types of cells (or entities) and the ECM (or matrix) that has been completely produced and arranged by some of these cells (or entities). “Living Tissue Matrix (LTM)” as used herein means, a 3-dimensional tissue (or matrix) that is capable of being transformed into a more complex tissue (or matrix) or a completely different type of tissue (or matrix) that consists of cells (or entities) and the ECM (or matrix) that has been completely produced and arranged by these cells (or entities).

“Living Tissue Equivalent (LTE)” as used herein means a construct containing living cells that intends to mimic a certain type of native tissue. This construct can be produced by any means in vitro, including by the use of artificial scaffolds.

“Culturing the cells in order to differentiate them” as used herein, means conditions that facilitate, aid, further or in any way allow the development of three-dimensional tissue growth. Conditions may include use of specific media, growth factors, minerals, incubation temperature, cell density, aeration, agitation, use of ALS “molds” to shape and contain growth of desired tissue, use of sub-atmospheric pressure chambers such as Synthecon's near-zero-gravity incubator systems (such as HARVs and STLVs) for growth of desired tissue, use of micro-carrier beads, use of natural or biodegradable scaffolds, implanting a non-fibroblast-seeded autogenic living scaffold within an in vivo site such as in an organ or tissue such as connective, epithelial, muscle, and/or nerve tissue.

“Genetically engineered” as used herein means that a cell or entity, by human manipulation such as chemical, physical, stress-induced, or other means, has undergone mutation and selection; or that an exogenous nucleic acid has actually been introduced to the cell or entity through any standard means, such as transfection; such that the cell or entity has acquired a new characteristic, phenotype, genotype, and/or gene expression product, including but not limited to a gene marker, a gene product, and/or a mRNA, to endow the original cell or entity, at a genetic level, with a function, characteristic, or genetic element not present in non-genetically engineered, non-selected counterpart cells or entities.

### PREFERRED EMBODIMENTS

The matrix-producing cell is cultured in a vessel suitable for animal cell or tissue culture, such as a culture dish, flask, or roller-bottle, which allows for the formation of a three-dimensional tissue-like structure. Suitable cell growth surfaces on which the cells can be grown can be any biologically compatible material to which the cells can adhere and provide an anchoring means for the cell-matrix construct to form. Materials such as glass; stainless steel; polymers, including polycarbonate, polystyrene, polyvinyl chloride, polyvinylidene, polydimethylsiloxane, fluoropolymers, and fluorinated ethylene propylene; and silicon substrates, including fused silica, polysilicone, or silicon crystals may be used as a cell growth surfaces.

While the tissue construct of the invention may be grown on a solid cell growth surface, a cell growth surface with pores that communicate both top and bottom surfaces of the membrane to allow bilateral contact of the medium to the developing tissue construct or for contact from only below the culture is preferred. Bilateral contact allows medium to contact both the top and bottom surfaces of the developing construct for maximal surface area exposure to the nutrients contained in the medium. Medium may also contact only the bottom of the forming cultured tissue construct so that the top surface may be exposed to air, as in the development of a cultured skin construct. The preferred culture vessel is one that utilizes a carrier insert, a culture-treated permeable member such as a porous membrane that is suspended in the culture vessel containing medium. Typically, the membrane is secured to one end of a tubular member or framework that is inserted within and interfaces with a base, such as a petri or culture dish that can be covered with a lid. When these types of culture vessels are employed, the tissue-construct is produced on one surface of the membrane, preferably the top, upwardly facing surface and the culture is contacted by cell media on both top and bottom surfaces. The pores in the growth surface allow for the passage of culture media for providing nutrients to the underside of the culture through the membrane, thus allowing the cells to be fed bilaterally or solely from the bottom side. A preferred pore size is one that is small enough that it does not allow for the growth of cells through the membrane, yet large enough to allow for free passage of nutrients contained in culture medium to the bottom surface of the cell-matrix construct, such as by capillary action. Preferred pore sizes are about less than 3 microns but range between about 0.1 microns to about 3 microns, more preferably between about 0.2 microns to about 1 micron and most preferably about 0.4 micron to about 0.6 micron sized pores are employed. In the case of human dermal fibroblasts, the most preferred material is polycarbonate having a pore size is between about 0.4 to about 0.6 microns. The maximum pore size depends not only on the size of the cell but also the ability of the cell to alter its shape and pass through the membrane. It is important that the tissue-like construct adheres to the surface but does not incorporate or envelop the substrate so it is removable from it such as by peeling with minimal force. The size and shape of the tissue construct formed is dictated by the size of the vessel surface or membrane on which it grown. Substrates may be round or angular or shaped with rounded corner angles, or irregularly shaped. Substrates may also be flat or contoured as a mold to produce a shaped construct to interface with a wound or mimic the physical structure of native tissue. To account for greater surface areas of the growth substrate, proportionally more cells are seeded to the surface and a greater volume of media is needed to sufficiently bathe and nourish the cells. When the tissue construct is finally formed, whether it is a single layer cell-matrix construct or a bi-layer construct, it is removed by peeling from the membrane substrate before grafting to a patient.

The system for the production of the cell-matrix layer may be either static or may employ a perfusion means to the culture media. In the static system, the culture medium is still and relatively motionless as contrasted to the perfusion system where the medium is in motion. The perfusion of medium affects the viability of the cells and augments the development of the matrix layer. Perfusion means include, but are not limited to: using a magnetic stir bar or motorized impeller in the culture dish subjacent (below) or adjacent to the substrate carrier containing the culture membrane to stir the medium; pumping medium within or through the culture dish or chamber; gently agitating the culture dish on a shaking or rotating platform; or rolling, if produced in a roller bottle. Other perfusion means can be determined by one skilled in the art for use in the method of the invention.

Culture media formulations suitable for use in the present invention are selected based on the cell types to be cultured and the extracellular matrix to be produced. The culture medium that is used and the specific culturing conditions needed to promote cell growth, matrix synthesis, and viability will depend on the type of cell being grown.

The use of chemically defined culture media is preferred, that is, media free of undefined animal organ or tissue extracts, for example, serum, pituitary extract, hypothalamic extract, placental extract, or embryonic extract or proteins and factors secreted by feeder cells. In a most preferred embodiment, the media are free of undefined components and defined biological components derived from non-human sources. When the invention is carried out utilizing screened human cells cultured using chemically defined components derived from no non-human animal sources, the resultant tissue construct is a defined human tissue construct. Synthetic functional equivalents may also be added to supplement chemically defined media within the purview of the definition of chemically defined for use in the most preferred fabrication method. Generally, one of skill in the art of cell culture will be able to determine suitable natural human, human recombinant, or synthetic equivalents to commonly known animal components to supplement the culture media of the invention without undue investigation or experimentation.

The advantages in using such a construct in the clinic is that the concern of adventitious animal or cross-species virus contamination and infection is diminished. In the testing scenario, the advantages of a chemically defined construct is that when tested, there is no chance of the results being confounded due to the presence of the undefined components.

Culture medium is comprised of a nutrient base usually further supplemented with other components. The skilled scientist can determine appropriate nutrient bases in the art of animal cell culture with reasonable expectations for successfully producing a tissue construct of the invention. Many commercially available nutrient sources are useful on the practice of the present invention. These include commercially available nutrient sources which supply inorganic salts, an energy source, amino acids, and B-vitamins such as Dulbecco's Modified Eagle's Medium (DMEM); Minimal Essential Medium (MEM); M199; RPMI 1640; Iscove's Modified Dulbecco's Medium (EDMEM). Minimal Essential Medium (MEM) and M199 require additional supplementation with phospholipid precursors and non-essential amino acids. Commercially available vitamin-rich mixtures that supply additional amino acids, nucleic acids, enzyme cofactors, phospholipid precursors, and inorganic salts include Ham's F-12, Ham's F-10, NCTC 109, and NCTC 135. Albeit in varying concentrations, all basal media provide a basic nutrient source for cells in the form of glucose, amino acids, vitamins, and inorganic ions, together with other basic media components. The most preferred base medium of the invention comprises a nutrient base of either calcium-free or low calcium Dulbecco's Modified Eagle's Medium (DMEM), or, alternatively , DMEM and Ham's F-12 between a 3-to-1 ratio to a 1-to-3 ratio, respectively.

The base medium is supplemented with components such as amino acids, growth factors, and hormones. Defined culture media for the culture of cells of the invention are described in U.S. Pat. No. 5,712,163 and in International PCT Publication No. WO 95/31473 the disclosures of which are incorporated herein by reference. Other media are known in the art such as those disclosed in Ham and McKeehan, Methods in Enzymology, 58:44-93 (1979), or for other appropriate chemically defined media, in Bottenstein et al., Methods in Enzymology, 58:94-109 (1979). In the preferred embodiment, the base medium is supplemented with the following components known to the skilled artisan in animal cell culture: insulin, transferrin, triiodothyronine (T3), and either or both ethanolamine and o-phosphoryl-ethanolamine, wherein concentrations and substitutions for the supplements may be determined by the skilled artisan.

Insulin is a polypeptide hormone that promotes the uptake of glucose and amino acids to provide long term benefits over multiple passages. Supplementation of insulin or insulin-like growth factor (IGF) is necessary for long term culture as there will be eventual depletion of the cells' ability to uptake glucose and amino acids and possible degradation of the cell phenotype. Insulin may be derived from either animal, for example bovine, human sources, or by recombinant means as human recombinant insulin. Therefore, human insulin would qualify as a chemically defined component not derived from a non-human biological source. Insulin supplementation is advisable for serial cultivation and is provided to the media at a wide range of concentrations. A preferred concentration range is between about 0.1 μg/ml to about 500 μg/ml, more preferably at about 5 μg/ml to about 400 μg/ml, and most preferably at about 375 μg/ml. Appropriate concentrations for the supplementation of insulin-like growth factor, such as IGF-1 or IGF-2, may be easily determined by one of skill in the art for the cell types chosen for culture.

Transferrin is in the medium for iron transport regulation. Iron is an essential trace element found in serum. As iron can be toxic to cells in its free form, in serum it is supplied to cells bound to transferrin at a concentration range of preferably between about 0.05 to about 50 μg/ml, more preferably at about 5 μg/ml.

Triiodothyronine (T3) is a basic component and is the active form of thyroid hormone that is included in the medium to maintain rates of cell metabolism. Truodothyronine is supplemented to the medium at a concentration range between about 0 to about 400 pM, more preferably between about 2 to about 200 pM and most preferably at about 20 pM.

Either or both ethanolamine and o-phosphoryl-ethanolamine, which are phospholipids, are added whose function is an important precursor in the inositol pathway and fatty acid metabolism. Supplementation of lipids that are normally found in serum is necessary in a serum-free medium. Ethanolamine and o-phosphoryl-ethanolamine are provided to media at a concentration range between about 10⁻⁶ to about 10⁻² M, more preferably at about 1×10⁻⁴ M.

Throughout the culture duration, the base medium is additionally supplemented with other components to induce synthesis or differentiation or to improve cell growth such as hydrocortisone, selenium, and L-glutamine.

Hydrocortisone has been shown in keratinocyte culture to promote keratinocyte phenotype and therefore enhance differentiated characteristics such as involucrin and keratinocyte transglutaminase content (Rubin et al., J. Cell PhysioL, 138:208-214 (1986)). Therefore, hydrocortisone is a desirable additive in instances where these characteristics are beneficial such as in the formation of keratinocyte sheet grafts or skin constructs. Hydrocortisone may be provided at a concentration range of about 0.01 ug/ml to about 4.0 μg/ml, most preferably between about 0.4 μg/ml to 16 ug/ml.

Selenium is added to serum-free media to resupplement the trace elements of selenium normally provided by serum. Selenium may be provided at a concentration range of about 10⁻⁹ M to about 10⁻⁷ M; most preferably at about 5.3×10⁻⁸ M.

The amino acid L-glutamine is present in some nutrient bases and may be added in cases where there is none or insufficient amounts present. L-glutamine may also be provided in stable form such as that sold under the mark, GlutaMAX-1™ (Gibco BRL, Grand Island, N.Y.). GlutaMAX-1™ is the stable dipeptide form of L-alanyl-L-glutamine and may be used interchangeably with L-glutamine and is provided in equimolar concentrations as a substitute to L-glutamine. The dipeptide provides stability to L-glutamine from degradation over time in storage and during incubation that can lead to uncertainty in the effective concentration of L-glutamine in medium. Typically, the base medium is supplemented with preferably between about 1 mM to about 6 mM, more preferably between about 2 mM to about 5 mM, and most preferably 4 mM L-glutamine or GlutaMAX-1™.

Growth factors such as epidermal growth factor (EGF) may also be added to the medium to aid in the establishment of the cultures through cell scale-up and seeding. EGF in native form or recombinant form may be used. Human forms, native or recombinant, of EGF are preferred for use in the medium when fabricating a skin equivalent containing no non-human biological components. EGF is an optional component and may be provided at a concentration between about 1 to 15 ng/mL, more preferably between about 5 to 10 ng/mL.

The medium described above is typically prepared as set forth below. However, it should be understood that the components of the present invention may be prepared and assembled using conventional methodology compatible with their physical properties. It is well known in the art to substitute certain components with an appropriate analogous or functionally equivalent acting agent for the purposes of availability or economy and arrive at a similar result. Naturally occurring growth factors may be substituted with recombinant or synthetic growth factors that have similar qualities and results when used in the performance of the invention.

Media in accordance with the present invention are sterile. Sterile components are bought sterile or rendered sterile by conventional procedures, such as filtration, after preparation. Proper aseptic procedures were used throughout the following Examples. DMEM and F-12 are first combined and the individual components are then added to complete the medium. Stock solutions of all components can be stored at −20° C., with the exception of nutrient source that can be stored at 4° C. All stock solutions are prepared at 500× final concentrations listed above. A stock solution of insulin, transferrin and triiodothyronine (all from Sigma) is prepared as follows: triiodothyronine is initially dissolved in absolute ethanol in IN hydrochloric acid (HCI) at a 2:1 ratio. Insulin is dissolved in dilute HCI (approximately 0.1N) and transferrin is dissolved in water. The three are then mixed and diluted in water to a 500× concentration. Ethanolamine and o-phosphoryl-ethanolamine are dissolved in water to 500× concentration and are filter sterilized. Progesterone is dissolved in absolute ethanol and diluted with water. Hydrocortisone is dissolved in absolute ethanol and diluted in phosphate buffered saline (PBS). Selenium is dissolved in water to 500× concentration and filter sterilized. EGF is purchased sterile and is dissolved in PBS. Adenine is difficult to dissolve but may be dissolved by any number of methods known to those skilled in the art. Serum albumin may be added to certain components in order to stabilize them in solution and are presently derived from either human or animal sources. For example, human serum albumin (HSA) or bovine serum albumin (BSA) may be added for prolonged storage to maintain the activity of the progesterone and EGF stock solutions. The medium can be either used immediately after preparation or, stored at 4° C. If stored, EGF should not be added until the time of use.

In order to form the cell-matrix layer by the culture of matrix-producing cells, the medium is supplemented with additional agents that promote matrix synthesis and deposition by the cells. These supplemental agents are cell-compatible, defined to a high degree of purity and are free of contaminants. The medium used to produce the cell-matrix is termed “matrix production medium”.

To prepare the matrix production medium, the base medium is supplemented with an ascorbate derivative such as sodium ascorbate, ascorbic acid, or one of its more chemically stable derivatives such as L-ascorbic acid phosphate magnesium salt n-hydrate. Ascorbate is added to promote hydroxylation of proline and secretion of procollagen, a soluble precursor to deposited collagen molecules. Ascorbate has also been shown to be an important cofactor for post-translational processing of other enzymes as well as an upregulator of type I and type III collagen synthesis.

While not wishing to be bound by theory, supplementing the medium with amino acids involved in protein synthesis conserves cellular energy by not requiring the cells produce the amino acids themselves. The addition of proline and glycine is preferred as they, as well as the hydroxylated form of proline, hydroxyproline, are basic amino acids that make up the structure of collagen.

While not required, the matrix-production medium is optionally supplemented with a neutral polymer. The cell-matrix constructs of the invention may be produced without a neutral polymer, but again not wishing to be bound by theory, its presence in the matrix production medium may collagen processing and deposition more consistently between samples. One preferred neutral polymer is polyethylene glycol (PEG), which has been shown to promote in vitro processing of the soluble precursor procollagen produced by the cultured cells to matrix deposited collagen. Tissue culture grade PEG within the range between about 1000 to about 4000 MW (molecular weight), more preferably between about 3400 to about 3700 MW is preferred in the media of the invention. Preferred PEG concentrations are for use in the method may be at concentrations at about 5% w/v or less, preferably about 0.01% w/v to about 0.5% w/v, more preferably between about 0.025% w/v to about 0.2% w/v, most preferably about 0.05% w/v. Other culture grade neutral polymers such dextran, preferably dextran T-40, or polyvinylpyrrolidone (PVP), preferably in the range of 30,000-40,000 MW, may also be used at concentrations at about 5% w/v or less, preferably between about 0.01% w/v to about 0.5% w/v, more preferably between about 0.025% w/v to about 0.2% w/v, most preferably about 0.05% w/v. Other cell culture grade and cell-compatible agents that enhance collagen processing and deposition may be ascertained by the skilled routineer in the art of mammalian cell culture.

When the cell producing cells are confluent, and the culture medium is supplemented with components that assist in matrix synthesis, secretion, or organization, the cells are said to be stimulated to form a tissue-construct comprised of cells and matrix synthesized by those cells. Therefore, a preferred matrix production medium formulation comprises: a base 3:1 mixture of Dulbecco's Modified Eagle's Medium (DMEM) (high glucose formulation, without L-glutamine) and Hams F-12 medium supplemented with either 4 mM L-glutamine or equivalent, 5 ng/ml epidermal growth factor, 0.4 μg/ml hydrocortisone, 1×10⁻⁴ M ethanolamine, 1×10⁻⁴ M o-phosphoryl-ethanolamine, 5 μg/ml insulin, 5 μg/ml transferrin, 20 pM triiodothyronine, 6.78 ng/ml selenium, 50 ng/ml L-ascorbic acid, 0.2 μg/ml L-proline, and 0.1 μg/ml glycine. To the production medium, other pharmacological agents may be added to the culture to alter the nature, amount, or type of the extracellular matrix secreted. These agents may include polypeptide growth factors, transcription factors or inorganic salts to up-regulate collagen transcription. Examples of polypeptide growth factors include transforming growth factor-beta 1 (TGF-β1) and tissue-plasmmogen activator (TPA), both of which are known to upregulate collagen synthesis. Raghow et al., Journal of Clinical Investigation, 79:1285-1288 (1987); Pardes et al., Journal of Investigative Dermatology, 100:549 (1993). An example of an inorganic salt that stimulates collagen production is cerium. Shivakumar et al., Journal of Molecular and Cellular Cardiology 24:775-780 (1992).

The cultures are maintained in an incubator to ensure sufficient environmental conditions of controlled temperature, humidity, and gas mixture for the culture of cells. Preferred conditions are between about 34° C. to about 38° C., more preferably 37±1° C. with an atmosphere between about 5-10±1% CO₂ and a relative humidity (Rh) between about 80-90%.

Once sufficient cell numbers have been obtained, cells are harvested and seeded onto a suitable culture surface and cultured under appropriate growth conditions to form a confluent sheet of cells. In the preferred embodiment, the cells are seeded on a porous membrane that is submerged to allow medium contact from below the culture through the pores and directly above. Preferably, cells are suspended in either base or growth media and are seeded on the cell culture surface at a density between about 1×10⁵ cells/cm² to about 6.6×10⁵ cells/cm², more preferably between about 3×10⁵ cells/cm² to about 6.6×10⁵ cells/cm², and most preferably at about 6.6×10⁵ cells/cm² (cells per square centimeter area of the surface). Cultures are cultured in growth medium to establish the culture and are cultured to between about 80% to 100% confluence at which time they are induced chemically by changing the medium to matrix production medium in order to upregulate the synthesis and secretion of extracellular matrix. In an alternate method, cells are seeded directly in production media to eliminate the need to change from the basic media to the production media but it is a method that requires higher seeding densities.

During the culture, the cells organize the secreted matrix molecules to form a three dimensional tissue-like structure but do not exhibit significant contractile forces to cause the forming cell-matrix construct to contract and peel itself from the culture substrate. Media exchanges are made every two to three days with fresh matrix production medium and with time, the secreted matrix increases in thickness and organization. The time necessary for creating a cell-matrix construct is dependent on the ability of the initial seeding density, the cell type, the age of the cell line, and the ability of the cell line to synthesize and secrete matrix.

When fully formed, the constructs of the invention have bulk thickness due to the fibrous matrix produced and organized by the cells; they are not ordinary confluent or overly confluent cell cultures where the cells may be loosely adherent to each other. The fibrous quality gives the constructs cohesive tissue-like properties unlike ordinary cultures because they resist physical damage, such as tearing or cracking, with routine handling in a clinical setting. In the fabrication of a cultured dermal construct, the cells will form an organized matrix around themselves on the cell culture surface preferably at least about 30 microns in thickness or more, more preferably between about 60 to about 120 microns thick across the surface of the membrane; however, thicknesses have been obtained in excess of 120 microns and are suitable for use in testing or clinical applications where such greater thicknesses are needed.

Optionally, mixed cell populations of two or more cell types may be cultured together during the formation of a tissue construct of the invention provided that at least one of the cell types used is capable of synthesizing extracellular matrix. The second cell type may be one needed to perform other tissue functions or to develop particular structural features of the tissue construct.

The production of the matrix in vitro in accordance with the present invention has shown to mimic several of the processes exhibited in production of matrix as well as repair of matrix in vivo.

The following examples are provided to better explain the practice of the present invention and should not be interpreted in any way to limit the scope of the present invention.

While the materials from which the module can be formed can generally be any moldable or otherwise formable material, the surface of the culture chamber, as well as any other surfaces of the module that may come into contact with the cells, nutrients, growth factors, or any other fluids or biochemicals that may contact the cells, should be of a suitable sterilizable, biocompatible material. In one particular embodiment, the membrane is formed so as to encourage cell anchorage at its surfaces.

The culture chamber can generally be of a shape and size so as to cultivate living cells within the chamber. In one preferred embodiment, culture chamber can be designed to accommodate a biomaterial scaffold within the culture chamber, while ensuring adequate nutrient flow throughout a cellular construct held in the culture chamber. For instance, a culture chamber can be between about 0.1 mm and about 10 mm in cross section. In another embodiment, the culture chamber can be greater than about 1 mm in any cross sectional direction. For instance, the chamber can be cylindrical in shape and about 5 mm in both cross sectional diameter and height. The shape of culture chamber is not critical to the invention, as long as flow can be maintained throughout a cellular construct held in the chamber.

The term “cell anchorage” as utilized herein refers to one or more articles, such as the membrane, upon which cells can attach and develop. For instance, the term “cell anchorage” can refer to a single continuous scaffold, multiple discrete scaffolds, or a combination thereof. The terms “cell anchorage”, “cellular anchorage,” and “anchorage” are intended to be synonymous. Any suitable cell anchorage as is generally known in the art can be located in the culture chamber to provide sites for cells and to encourage the development of a three-dimensional cellular construct within the culture chamber.

For purposes of the present disclosure, the term continuous scaffold is herein defined to refer to a construct suitable for use as a cellular anchorage that can be utilized alone as a single, three- dimensional entity. A continuous scaffold is usually porous in nature and has a semi-fixed shape. Continuous scaffolds are well known in the art and can be formed of many materials, e.g., coral, collagen, calcium phosphates, synthetic polymers, and the like, and are usually pre-formed to a specific shape designed for the location in which they will be placed.

Continuous scaffolds are usually seeded with the desired cells through absorption and cellular migration, often coupled with application of pressure through simple stirring, pulsatile perfusion methods or application of centrifugal force.

Discrete scaffolds are smaller entities, such as beads, rods, tubes, fragments, or the like. When utilized as a cellular anchorage, a plurality of identical or a mixture of different discrete scaffolds can be loaded with cells and/or other agents and located within a void where the plurality of entities can function as a single cellular anchorage device. Exemplary discrete scaffolds suitable for use in the present invention that have been found particularly suitable for use in vivo are described further in U.S. Pat. No. 6,991,652 to Burg, which is incorporated herein by reference. A cellular anchorage formed of a plurality of discrete scaffolds can be preferred in certain embodiments of the present invention as discrete scaffolds can facilitate uniform cell distribution throughout the anchorage and can also allow good flow characteristics throughout the anchorage as well as encouraging the development of a three-dimensional cellular construct.

In one embodiment, for instance when considering a cellular anchorage including multiple discrete scaffolds, the anchorage can be seeded with cells following assembly and sterilization of the system. For example, an anchorage including multiple discrete scaffolds can be seeded in one operation or several sequential operations. Optionally, the anchorage can be pre-seeded, prior to assembly of the system. In one embodiment, the anchorage can include a combination of both pre-seeded discrete scaffolds and discrete scaffolds that have not been seeded with cells prior to assembly of the system.

The good flow characteristics possible throughout a plurality of discrete scaffolds can also provide for good transport of nutrients to and waste from the developing cells, and thus can encourage not only healthy growth and development of the individual cells throughout the anchorage, but can also encourage development of a unified three-dimensional cellular construct within the culture chamber.

The materials that are used in forming an anchorage can generally be any suitable biocompatible material. In one embodiment, the materials forming a cellular anchorage can be biodegradable. For instance, a cellular anchorage can include biodegradable synthetic polymeric scaffold materials such as, for example, polylactide, chondroitin sulfate (a proteoglycan component), polyesters, polyethylene glycols, polycarbonates, polyvinyl alcohols, polyacrylamides, polyamides, polyacrylates, polyesters, polyetheresters, polymethacrylates, polyurethanes, polycaprolactone, polyphophazenes, polyorthoesters, polyglycolide, copolymers of lysine and lactic acid, copolymers of lysine-RGD and lactic acid, and the like, and copolymers of the same. Optionally, an anchorage can include naturally derived biodegradable materials including, but not limited to chitosan, agarose, alginate, collagen, hyaluronic acid, and carrageenan (a carboxylated seaweed polysaccharide), demineralized bone matrix, and the like, and copolymers of the same.

A biodegradable anchorage can include factors that can be released as the scaffold(s) degrade. For example, an anchorage can include within or on a scaffold one or more factors that can trigger cellular events. According to this embodiment, as the scaffold(s) forming the cellular anchorage degrades, the factors can be released to interact with the cells.

In those embodiments including a cellular anchorage formed with a plurality of discrete scaffolds, a retaining mesh can also be located on, or be part of, the membrane of the culture chamber. The retaining mesh can be formed of any suitable biocompatible material, such as polypropylene, for example, and can line at least a portion of a culture chamber, so as to prevent material loss during media perfusion of the culture chamber.

A porous retaining mesh can generally have a porosity of a size of between about 10 microns and about 150 microns.

Upon assembly of the system, two (or more) culture chambers can be aligned so as to be immediately adjacent to one another. Between two adjacent culture chambers can be a gasket including a permeable membrane portion. The membrane portion of gasket can be aligned between the culture chambers and can have a porosity that can allow biochemical materials, for instance growth factors produced by a cell in one chamber, to pass through the membrane and into the adjacent chamber, where interaction can occur between the material produced in the first chamber and the cells contained in the second chamber. The membrane porosity can be small enough to prevent passage of the cells or cell extensions from one chamber to another. In particular, the membrane porosity can be predetermined so as to discourage physical contact between the cells held in adjacent chambers, and thus maintain isolation of the cell types.

Suitable porosity for a membrane can be determined based upon specific characteristics of the system, for instance the nature of the cells to be cultured within the chamber(s). Such determination is well within the ability of one of ordinary skill in the art and thus is not discussed at length herein.

Physical isolation of cellular contents of adjacent chambers can also be encouraged through selection of membrane materials. For instance, materials used to form the membrane can be those that encourage anchorage of cells onto the membrane.

In another embodiment the cells contained in a culture chamber can be maintained at a distance from the membrane to discourage physical contact between cells held in adjacent culture chambers. For instance retaining mesh can be located between a cell anchorage held in a culture chamber and the membrane located between two adjacent chambers.

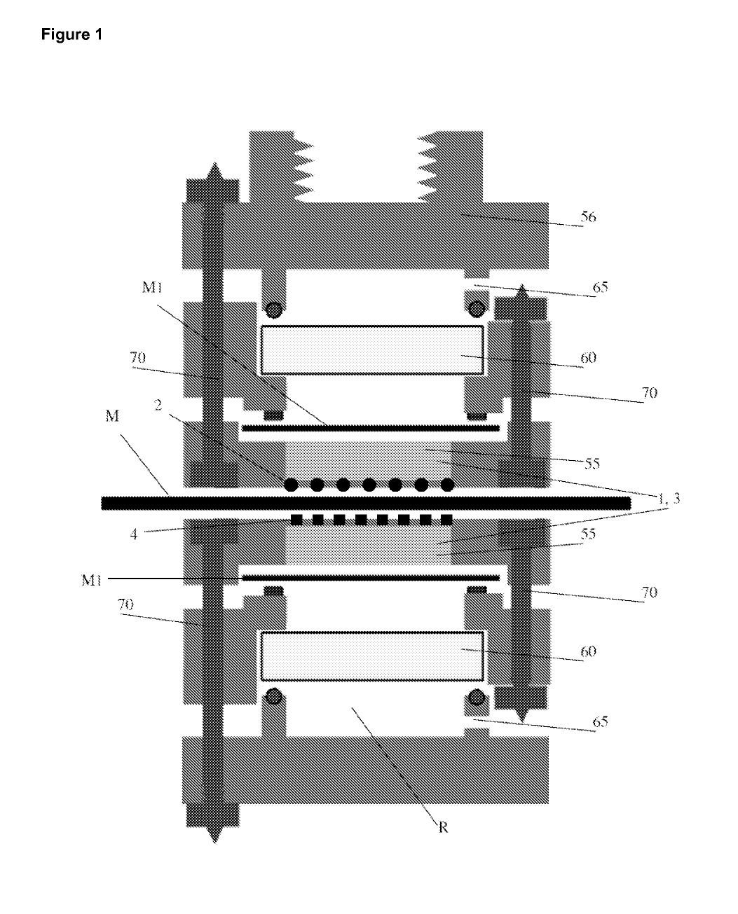

Each culture chamber of the system can include the capability for independent flow control through the chamber. For example, and referring again to FIG. 1, each individual culture chamber can include an inlet and an outlet (not shown) through which medium can flow.

### EXAMPLE

The present example serves to demonstrate that the bioreactor system of the present invention is suitable for generating ECM and soluble factors pertaining to a desired tissue type. As appears from the above description as well as the appending claims the present invention utilizes cells of a desired tissue to differentiate BMMSCs into cells of that tissue, whereby ECM and soluble factors are produced.

FIG. 1 is a detailed cross-sectional drawing of a bioreactor according to the present invention showing inter alia air inlets 65 and a base part 56 whereupon the bioreactor is mountable. The air inlet 65 supplies a rear cavity R with fresh air. Optionally, the air inlet 65 is connected to or embedded in a gas supply unit (not shown) for providing a controlled atmosphere in the rear cavity R. Thereby the aeration of the cells in the incubation cavity 55 will also be controlled via the air flow passing through a humidity chamber 60.

In order to hold the various parts of the bioreactor firmly together, appropriate fastening means are provided. As shown in FIG. 1, through-going assembly screws 70 keep the various parts together. The incubation cavity 55 of FIG. 1 has a substantially cylindrical shape having a diameter of the cavity 55 in the range from 4 to 20 mm, such as 6, 8, 10, 12, 14, 16, or 18 mm. The depth of the cavity 55 may be in the range from 2 to 6 mm, such as 2.5, 3, 3.5, 4, 4.5 or 5 mm. Thus, the volume of the cavity 55 is in the range from about 0.03 to 2 ml. Some preferred values of the depth and the diameter, respectively, are 4 mm and 18 mm (resulting in a fluid volume of about 1 ml), 3 mm and 10 mm (resulting in a fluid volume of about 0.24 ml), and 3 mm and 7.5 mm (resulting in a fluid volume of about 0.15 ml). The bioreactor 55 is adapted for rotation around a horizontal, rotational axis by associated rotation means (not shown). The base part 56 has a threaded portion 56a in order to facilitate easy and flexible mounting on such rotation means. Typically, the rotational axis is substantially coincident with a central axis through the incubation cavity 55.

In accordance with the figure there is provided regular growth media (1), BMMSC cultured on Human liver cells ECM (2), human liver cells conditioned media (3), and human liver cells cultured under the insert (4). The BMMSC and human liver cells are separated from each other by the membrane M.

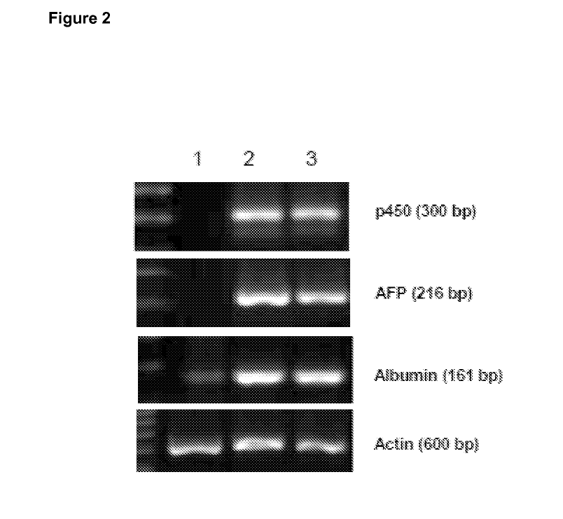

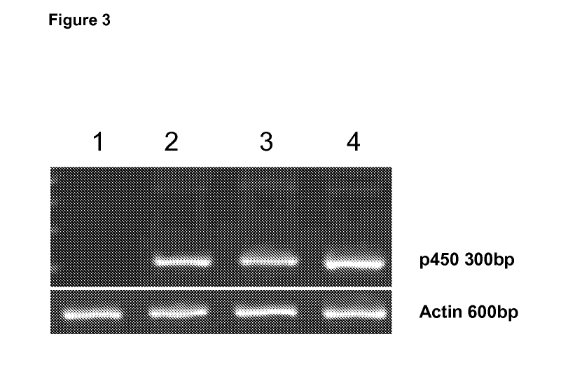

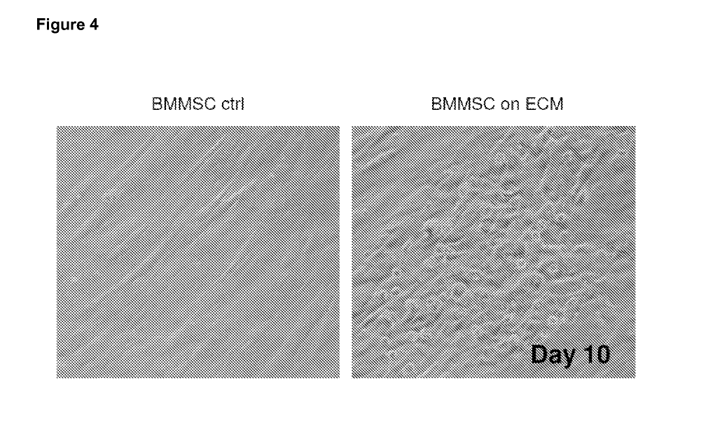

The present example illustrates the applicability of the present invention to differentiate human BMMSCs into hepatocytes thereby producing valuable human based ECM and soluble factors. Upon differentiation of the BMMSCs into hepatocytes the ECM and soluble factors are obtained by removing the differentiated cells leaving ECM and soluble factors; commonly denoted ECM extract. This extract may then be stored and later used as differentiation medium for the differentiation of BMMSCs into hepatocytes (ex vivo or in vivo). The differentiation of the human BMMSCs into hepatocytes is visualized in FIG. 4, whereas the biochemical changes associated therewith are shown in FIGS. 2 and 3.

Accordingly, this example illustrates how the method of the present invention works, namely to provide a human-based extracellular matrix extract and soluble factors of a liver tissue, comprising,
seeding human BMMSCs in a culture vessel in a first culture chamber,
providing liver cells (hepatocytes) to a second culture chamber, said liver cells comprising extracellular matrix resembling the extracellular matrix of the liver,
maintaining said BMMSCs and liver cells in a physically isolated state from one another; and allowing biochemical communication between them,
culturing the BMMSCs in order to differentiate them into liver cells, whereby the cells are stimulated to synthesise, secrete and organize extracellular matrix of liver tissue;
continued culturing of the BMMSCs until the cells have been differentiated into liver cells and have synthesized extracellular matrix and soluble factors, and
removing the differentiated liver cells to obtain the extracellular matrix extract and soluble factors of liver tissue.

In the following these steps are exemplified in more detail.

BMMSC Isolation and Culture:

Human BMMSC were isolated from 3-4ml of bone marrow aspirates using Ficoll density gradient. Cells were cultured in DMEM (1 g/L glucose) supplemented with 1% penicillin/streptomycin and 10% fetal bovine serum at 37C in 95% air with 5% carbon dioxide at 100% humidity. Medium was replenished every 3 days. Confluent cultures were passaged by trypsinization and gentle scraping. Cells at passage 5 were used in the experiments.

Human Hepatocyte Cell Culture:

Human hepatocytes were cultured in DMEM (1 g/L glucose) supplemented with 1% non essential amino acids, 1% penicillin/streptomycin, 1% glutamax and 10% fetal bovien serum at 37C in 95% air with 5% carbon dioxide at 100% humidity. Cells were cultured for a maximum of 15 days after which new cells were put in culture.

BMMSC Differentiation

Human BMMSCs were seeded in 6-well format inserts at a density of 30000 cells. In the compartment underneath the inserts, human liver cells were seeded at the same density. Cells were replenished with fresh growth media every 3 days. The media used to replenish the cells was each cell growth media. Cells were harvested 10 days post co-culture.

Human liver cells growth media: DMEM low glucose, non essential amino acids, fetal bovine serum, glutamax, penicillin/streptomycine.

BMMSC growth media: DMEM low glucose, fetal bovine serum, peniciline/streptomycine.

Preparation of ECM and Conditioned Media

Human liver cells were cultured in T75 flasks. After 48 h of cell counfluency, the media was removed and filtered using 0.2 um syringe filter and stored at −80C. The liver cells were then washed once after which a “removing reagent” was added to the cells ensuring total removal of cells while leaving the deposited ECM intact. The ECM was gently washed once with media (without FBS) to remove any traces of the removing agent and then the ECM was scraped in 2 ml of media (without FBS) and aliquoted and stored at −80C.

Differentiation of BMMSC into Hepatocytes by Culturing on Human Liver ECM

In order to test whether or not the liver ECM extract obtained in accordance with the above protocol cell culture flasks coated with human liver ECM extracts were seeded with BMMSC. Cells were allowed to interact with matrix for 10 days, RNA is extracted and hepatocyte differentiation markers monitored.

As shown in FIG. 2 hepatocyte markers induced in BMMSC: 1- human hepatocyte cells express the various liver markers. 2- On the other hand, undifferentiated BMMSC do not express liver markers. 3- When BMMSC differentiate into hepatocytes these cells start expressing liver specific markers: AFP and p450.

As can be derived from FIG. 3 the induction of p450 expression in ECM differentiated BMMSCs takes place, which verifies that the alleged differentiation has taken place.

Undifferentiated BMMSCs do not express p450 (1). After culturing BMMSCs on human liver cell ECM, p450 expression was induced (2) and increased induction was detected upon the treatment with 1mM phenobarbital for 1 h (3) and 4 h (4). House keeping gene was used to ensure equal loading of the samples.

FIG. 4 shows the morphology of differentiated BMMSCs cultivated on human liver cell ECM. BMMSCs cultured on plastic maintained their spindle shape morphology (right panel), where as when cultured on human liver ECM for 10 days these cells changed to round shaped cells (left panel).

1.-9. (canceled)

10. A method for producing human-based extracellular matrix and soluble factors of a desired human tissue, comprising,
seeding human stem cells in a culture vessel in a first culture chamber either as a suspension or adhered to beads,
providing cells of desired tissue to a second culture chamber, said cells of desired tissue providing extracellular matrix resembling the extracellular matrix of the desired tissue,
maintaining said stem cells and said cells of desired tissue in a physically isolated state from one another, while allowing biochemical communication between said first said cell type and said second cell type,
culturing the stem cells in order to differentiate them into cells of the desired tissue, whereby the cells are stimulated to synthesise, secrete and organize extracellular matrix;
continued culturing of the cells until the cells have been differentiated into cells of the desired tissue and have synthesized extracellular matrix and soluble factors, and
removing the cells to obtain the extracellular matrix extract and soluble factors of the desired tissue.

11. The method according to claim 10, wherein said biochemical communication is allowed via a semi-permeable membrane located between said first culture chamber and said second culture chamber.

12. The method of claim 10, wherein the desired tissue is selected from the group consisting of liver, pancreas, cartilage, and bone-marrow.

13. The method of claim 11, wherein the desired tissue is selected from the group consisting of liver, pancreas, cartilage, and bone-marrow.

14. The method of claim 10, wherein the cells are removed by centrifuging the differentiated cells.

15. The method of claim 11, wherein the cells are removed by centrifuging the differentiated cells.

16. The method of claim 12, wherein the cells are removed by centrifuging the differentiated cells.

17. The method of claim 14, wherein the cells are genetically modified to produce a growth factor, hormone, peptide, or protein.

18. The method of claim 15, wherein the cells are genetically modified to produce a growth factor, hormone, peptide, or protein.

19. The method of claim 16, wherein the cells are genetically modified to produce a growth factor, hormone, peptide, or protein.
